(19) United States
(12) Patent Application Publication
(10) Pub. No.: US 2007/0003641 A1
(43) Pub. Date: Jan. 4, 2007

(54) Synergistic pharmaceutical compositions useful in prevention and treatment of beta-amyloid protein-induced disease

(76) Inventor: Darrick S. H. L. Kim, Schaumburg, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/453,224

(22) Filed: Jun. 14, 2006

Related U.S. Application Data
(60) Provisional application No. 60/690,812, filed on Jun. 15, 2005.
(60) Provisional application No. 60/739,797, filed on Nov. 23, 2005.

(51) Int. Cl. A61K 36/906; A61K 36/53; A61K 36/537; A61K 36/16; A61K 31/365; A61K 31/192

(52) U.S. Cl. 424/745; 424/756; 424/746; 424/752; 514/462; 514/568; 514/570; 514/678

(57) ABSTRACT

Disclosed are combinations of natural and synthetic turmeric, ginger, ginko biloba, sage, and rosemary compounds suitable for treatment of beta-amyloid-disease induced disease that have synergistic anti-βA peptide effects when members of the five groups of compounds are combined. Suitable members of the compounds include both natural compounds derived from extracts of each of Curcuma sp., Zingiber sp., Ginkgo biloba, Salvia sp., or Rosmarinus sp. as well as synthetic homologues and analogues of such natural compounds. Sage and rosemary derived compounds suitable alone for treatment of beta-amyloid induced disease is also described.

### CROSS REFERENCE TO RELATED APPLICATIONS

This application claims benefit of U.S. Provisional Application Serial No. 60/739,797 filed Nov. 23, 2005 and of U.S. Provisional Application Serial No. 60/690,812 filed Jun. 15, 2005, the disclosures of which are incorporated herein by reference in their entireties.

### BACKGROUND OF THE INVENTION

1. Field of the Invention

The invention relates to the use of an extract or a combination of plant extracts that contain natural product compounds from Curcuma longa (Zingiberaceae), and related Curcuma sp, Zingiber officinale (Zingiberaceae), and related Zingiber sp, Ginkgo biloba (Ginkgoaceae), and Salvia officinalis (Lamiaceae), and related Salivia sp. and Rosmarinus officinalis (Labiatae), and related Rosmarinus sp. for the prevention and treatment of beta-amyloid-induced disease. More particularly, the invention relates to compositions of combinations of plant extracts and the natural compounds of said plants and synthetic analogues and homologues that protect neuronal cells from beta-amyloid insult for use in preventing and treating beta-amyloid-induced disease.

2. Description of Related Technology

Alzheimer's disease (AD) is the most common cause of progressive cognitive dysfunction. AD affects approximately four million Americans and causes more than 100,000 deaths each year, with a total annual cost approaching $100 billion. It is estimated that by the year 2020, 14 million Americans will be afflicted by the disease. See Carr et al., Am J Med 103, 3S (1997) and Shastry, Am J Med Sci 315, 266 (1998). Furthermore, AD has a profound effect on the millions of family members and other loved ones who provide most of the care for people having this disease. Unfortunately, the cure for AD has not yet been discovered.

The principal pathological characteristics of AD are senile plaques and neurofibrillary tangles (NTFs). Senile plaques are extracellular deposits principally composed of insoluble aggregates of beta-amyloid (βA), that are infiltrated by reactive microglia and astrocytes. See Seidl et al., Neurosci. Lett 232, 49 (1997), Yan et al., Nature 382, 685 (1997), Goedert, Trends Neurosci 16, 460 (1993), Haass et al., Cell 7, 1039 (1994), Trojanowski et al., Am J Pathol 144, 449 (1994), Davis et al., Biochem Biophys Res Commun 189, 1096 (1992), Pike et al., Neuroscience 13, 1676 (1993), Hensley et al., Proc Natl Acad Sci USA 91, 3270 (1994), Behl et al., Cell 77, 817 (1994), Meda et al., Nature 374, 647 (1995), and Klegeris et al., Biochem Biophys Res Commun 199, 984 (1994). Plaques are diffusely distributed throughout the cerebral cortex of AD patients, and are the neuropathologic hallmark of the disease. See Seidl et al., Neurosci Lett 232, 49 (1997), Yan et al., Nature 382, 685 (1997), Goedert, Trends Neurosci 16, 460 (1993), Haass et al., Cell 7, 1039 (1994) and Trojanowski et al., Am J Pathol 144, 449 (1994). These plaques or βA fibril deposits are believed to be responsible for the pathology of a number of neurodegenerative diseases including, but not limited to, Alzheimer's disease. NTFs are intraneuronal accumulation of paired helical filaments composed mainly of an abnormal form of tau protein, that is a microtubule associated phosphoprotein which can promote microtubule formation. See Goedert, Trends Neurosci 16, 460 (1993), Haass et al., Cell 7, 1039 (1994) and Trojanowski et al., Am J Pathol 144, 449 (1994). In the AD brain, the tau protein in NFTs is hyperphosphorylated (See Ihara et al., J Biochem 99, 1807 (1986)), a condition which has been suggested to contribute to the destabilization of microtubule network, thereby impairing axonal network, and eventually causing neuronal death. See Trojanowski et al., FASEB J 9, 1570 (1995). NTFs occur primarily in medial temporal lobe structures (hippocampus, entorhinal cortex, and amygdala), and NTFs density appears to correlate with dementia severity.

Senile plaques and NTFs appear to be involved in cerebral amyloid angiopathy, consequent neuronal loss, and cerebral atrophy leading to dementia. Although research findings suggest that both plaques and NTFs are involved in disrupting nerve cell functions, the mechanisms that lead to the pathology are not clearly understood.

βA has been suggested as one of the major causes of AD. βA was shown to exert direct toxic effects on neurons and to inhibit neurite growth in vitro in a dose dependent manner. Thus, therapeutic approaches that can modulate βA toxicity have been hypothesized to represent important methods for controlling the onset of AD. It is envisioned that if neuronal cells can be protected from βA/senile plaque-induced toxicity, the onset of AD may be delayed or prevented. Current pharmacological approaches related to AD preventive and neuroprotective interventions include antioxidant therapy (See Lucca et al., Brain Res 764, 293 (1997), Pike et al., J Neurochem 69, 1601 (1997), Manelli et al., Brain Res Bull 38, 569 (1995), Parnetti et al., Drugs 53, 752 (1997), Zhou et al., J Neurochem 67, 1419 (1996), Kumar et al., Int J Neurosci 79, 185 (1994), Preston et al., Neurosci Lett 242, 105 (1998), and Tatton et al., Neurology 47, S171 (1996)), acetylcholinesterase inhibitors (See Hoshi et al., J Biol Chem 272, 2038 (1997), Maurice et al., Brain Res 706, 181 (1996), Harkany et al., Brain Res 695, 71 (1995), and Lahiri et al., J Neurosci Res 37, 777 (1994)), nicotinic and muscarinic agonists (See Maurice et al., Brain Res 706, 181 (1996), and Kihara et al., Brain Res 792, 331 (1998)), estrogen (See Ihara et al., J Biochem 99, 1807 (1986), Henderson, Neurology 48 (5 Suppl. 7), S27 (1997), and Green et al., Neuroscience 84, 7 (1998)), nerve growth factor (NGF) (See Hefti, Neurobiol Aging 15 (Suppl 2), S193 (1994), and Seiger et al., Behav Brain Res 57, 255 (1993)), calcium channel blockers (See Zhou et al., J Neurochem 67, 1419 (1996) and Friedlich et al., Neurobiol Aging 15, 443 (1994)), Zinc (See Cuajungco et al., Neurobiol Dis 4, 137 (1997)), sulfonated compounds (See Pollack et al., Neurosci Lett 197 211 (1995) and Lorenzo, et al., Ann NY Acad Sci 777, 89 (1996)), triaminopyridine nonopiate analgesic drug (See Muller et al., J Neurochem 68, 2371 (1997)), low molecular lipophilic compounds that can activate neurotrophic factor signaling pathway (See Mattson, Neurosci Biobehav Rev 21, 193 (1997)), and non-steroidal anti-inflammatory drugs such as ibuprofen and aspirin (See Parnetti et al., Drugs 53, 752 (1997), Beard et al., Mayo Clin Proc 73, 951 (1998), and Pasinetti et al., Neuroscience 87, 319 (1998)). Of particular interest to the present invention is the observation that an anti-βA protein antibody was shown to clear senile plaques and protect mutant PDAPP mice from the onset of AD. See St George-Hyslop et al., Nature 400, 116 (1999).

The generation of reactive oxygen intermediates (ROS) through oxidative stress caused by βA has been suggested to be the major pathway of the βA-induced cytotoxicity. See Klegeris et al., Biochem Biophys Res Comun 199, 984 (1994) and Lucca et al., Brain Res 764, 293 (1997). Senile plaques have been shown to exerts a cytotoxic effect on neurons by stimulating microglia to produce reactive oxygen species (ROS). See Seidl et al., Neurosci Lett 232, 49 (1997), Yan et al., Nature 382, 685 (1997), Goedert, Trends Neurosci 16, 460 (1993), Haass et al., Cell 7, 1039 (1994), Trojanowski et al., Am J Pathol 114, 449 (1994), Davis et al., Biochem Biophys Res Commun 189, 1096 (1992), Pike et al., Neuroscience 13, 1676 (1993), Hensley et al., Proc Natl Acad Sci USA 91, 3270 (1994), Behl et al., Cell 77, 817 (1994), Meda et al., Nature 374, 647 (1995) and Klegeris et al., Biochem Biophys Res Commun 199, 984 (1994). The damaging effect of ROS can be prevented by the free radical scavenging enzyme superoxide dismutase (SOD). See Thomas et al., Nature 380, 168 (1996) and Manelli et al., Brain Res Bull 38, 569 (1995).

Aging of synthetic βA for 7 to 14 days at 37° C. in modified Eagle's media was also demonstrated to cause neurotoxic free radical formation. See Friedlich et al., Neurobiol Aging 15, 443 (1994) and Puttfarcken et al., Exp Neurol 138, 73 (1996). However, aging βA in the presence of the media supplement B27, which contains antioxidants as well as other agents that provide protection against oxidative damage, has been shown to inhibited βA-induced neurotoxicity. See Thomas et al., Nature 380, 168 (1996) and Manelli et al., Brain Res Bull 38, 569 (1995).

In designing inhibitors of βA toxicity, it was found that neither the alteration of the apparent secondary structure of βA nor the prevention of βA aggregation is required to abrogate the cytotoxicity of βA. Nonethless, inducing changes in aggregation kinetics and in higher order structural characteristics of βA aggregate also proved to be effective in reducing βA toxicity. See Ghanta et al., J Biol Chem 271, 29525 (1996). Synthetic inhibitors that interact with βA was shown to completely block βA toxicity against PC12 cells, demonstrating that complete disruption of amyloid fibril formation is not necessary for abrogation of toxicity. It was also demonstrated that dipolar compounds such as phloretin and exifone that decrease the effective negative charge of membranes can prevent the association of βA to negatively charged lipid vesicles and thereby prevent the βA-induced cytotoxicity. See Yaar et al., J Clin Invest 100, 2333 (1997) and Hertel et al., Proc Natl Acad Sci USA 94 9412 (1997). These results suggest that βA toxicity can be mediated through a physicochemical interaction with cell membranes.

There is strong interest in discovering potentially valuable natural sources for drug development. One reasonable source of such natural products involves medicinal plants that have been in use throughout history for treating various ailments. Thus, the discovery of potentially valuable plants that can protect neurons from βA insult is of interest.

Curcuma longa (Zingiberaceae) has been used as curry spice and a well known constituent of Indonesian traditional medicine. See Nurfina et al., Eur J Med Chem 32, 321 (1997). One of the important constituents of turmeric is curcumin that has been known as a natural antioxidant with antitumor activity. See Ruby et al., Cancer Lett 94, 79 (1995). From turmeric, curcuminoids with antioxidant property have been demonstrated to protect neuronal cells from βA insult. See Kim DSHL et al., Neurosci Lett 303, 57 and Park S Y et al., J Nat Prod 65, 1227 (2002). A representative list of Curcuma sp. include C. longa, C. aromatica, C. domestica, C. xanthorrhiza, and C. zedoaria.

Zingiber officinale (Zingiberaceae) is one of the world's favorite spices, probably discovered in the tropics of Southeast Asia. Ginger has benefited humankind as a wonder drug since the beginning of recorded history. See Jitoe et al., J Agric Food Chem 40, 1337 (1992), Kikuzaki et al., J Food Sci 58, 1407 (1993) and Schulick, Herbal Free Press, Ltd. (1994). From ginger, shogaols with antioxidant property have also been demonstrated to protect neuronal cells from βA insult. See Kim et al., Planta Medica 68, 375 (2002). A representative list of Zingiber sp. include Z. officinale, Z. zerumbet, and Z. mioga.

Ginkgo (Ginkgo biloba (Ginkgoaceae)) is an herbal that has been used to treat neurologic ailment for thousand years as an Asian traditional medicine. Ginkgo leaf extract has shown to exhibit potent antioxidant activity and are widely used in the dietary supplement industry. The antioxidant activity of ginkgo has shown to be primarily contributed by diterpenes such as ginkgolides, bilobilide, flavonoids, and ginkgolic acids. See Hopia et al., J Agric Food Chem 44, 2030 (1996) and Nakatani et al., Agric Biol Chem 47, 353 (1983).

Sage (Salvia officinalis L. (Lamiaceae)) and Rosemary (Rosmarinus officinalis L. (Labiatae)) are spices widely used for flavoring and seasoning foods. These spices have shown to contain potent diterpenoid antioxidants such as carnosic acid, carnosol, rosmarinic acid, rosmanol, epirosmanol, rosmadial, isorosmanol etc. See Haraguchi et al., Planta Med 61, 333 (1995). Inatani et al, Agric Biol Chem 47: 521 (1983). Nakatani et al., Agric Biol Chem 48: 2081 (1984). Inatani et al., Agric Biol Chem 46: 1661 (1982). Wang et al., J Agric Food Chem 46: 2509 (1998). Wang et a., J Agric Food Chem 46: 4869 (1998).

Of interest to the present invention is the disclosure of co-owned U.S. Pat. No. 6,887,898 the disclosure of which is hereby incorporated by reference. This patent discloses in part numerous compounds isolated and/or derived from and/or are homologues and analogues of compounds, as well as their extracts, derived from Curcuma longa, Zingiber officinale, Ginkgo biloba, Salvia officinalis, and Rosmarinus officinalis, each of which each have independent activity against beta-Amyloid induced cytotoxicity including neurotoxicity.

Of further interest to the present application is the disclosure of co-owned and copending U.S. Provisional Application No. 60/690,812 filed Jun. 15, 2005 the disclosure of which is hereby incorporated herein which discloses synergistic combinations of compounds isolated and/or derived from and/or are homologues and analogues of compounds, as well as their extracts, derived from Curcuma longa, Zingiber officinale, and Ginkgo biloba.

### SUMMARY OF THE INVENTION

The present invention relates to the discovery that natural compounds present in rosemary and sage exhibit potent anti-βA peptide activity. The invention further provides novel synthetic compounds which are analogues or homologues of naturally occurring rosemary and sage compounds exhibit potent anti-βA peptide activity. Specifically, the invention provides compounds and pharmaceutical compositions capable of protecting neurons from βA peptide insult, and methods for treating βA protein-induced disease with the same.

The present invention is also related to the discovery that combinations of natural and synthetic turmeric, ginger, ginkgo biloba, sage, and rosemary compounds have synergistic anti-βA peptide effects when members of these five groups of compounds are combined. Specifically, the invention provides a pharmaceutical composition comprising at least at least two of a) a natural or synthetic turmeric compound having anti-βA peptide activity; b) a natural or synthetic ginkgo biloba compound having anti-βA peptide activity; c) a natural or synthetic ginger compound having anti-βA peptide activity; d) a natural or synthetic sage compound having anti-βA peptide activity; and e) a natural or synthetic rosemary compound having anti-βA peptide activity. Suitable members of the compounds include both natural compounds derived from extracts of each of Curcuma longa and related species, Zingiber officinale and related species, Ginkgo biloba, Salvia officinalis and related species, and Rosmarinus officinalis and related species but also include analogues and homologues of such natural compounds having anti-βA peptide biological activities (hereinafter “synthetic compounds”). Such synthetic compounds are in part disclosed in U.S. Pat. No. 6,887,898 the disclosure of which is hereby incorporated therein.

As used herein, synthetic turmeric, ginger, ginkgo biloba, sage, or rosemary compounds include chemically synthesized versions of naturally occurring turmeric sp., ginger sp., ginko biloba, sage sp., or rosemary sp. compounds respectively as well as analogues and homologues of such naturally occurring compounds which have anti-βA peptide activity. As used herein anti-βA peptide activity includes, but is not limited to, the ability to neutralize amyloid protein mediated cytotoxicity including neurotoxicity.

Thus, the present invention is directed to treating (which when used herein also includes preventing) βA-induced disease including beta-Amyloid induced cytotoxicity of Alzheimer's Disease (AD), and Down's syndrome. The invention also provides methods of treating beta-Amyloid induced ocular disease including, in particular, glaucoma and age-related macular degeneration (AMD) according to the methods described in co-owned and copending U.S. patent application Ser. No. 11/287,080 filed Nov. 23, 2005 [Attorney Docket No. 30443/41270] entitled “Methods for treatment of Beta-Amyloid Protein-Induced Ocular Disease” the disclosure of which is hereby incorporated by reference.

According to one aspect of the invention an extract or a combination of extracts containing natural compounds found in particular plants (as well as synthetic analogues and homologues thereof) as the major ingredients or components may be administered to protect cells from βA insult. Natural compounds that are suitable for use with the invention include, but are not limited to 4″-(3′″-methoxy-4′″-hydroxyphenyl)-2″-oxo-3″-enebutanyl 3-(3′-methoxy-4′hydroxyphenyl)propenoate (calebin-A) and 1,7-bis(4-hydroxy-3-methoxyphenyl)-1,4,6-heptatrien-3-one, and seven known compounds, 1,7-bis(4-hydroxy-3-methoxyphenyl)-1,6-heptadiene-3,5-dione (curcumin), 1-(4-hydroxy-3-methoxyphenyl)-7-(4-hydroxyphenyl)-1,6-heptadiene-3,5-dione (demethoxycurcumin), 1,7-bis(4-hydroxyphenyl)-1,6-heptadiene-3,5-dione (bisdemethoxycurcumin), 1-hydroxy-1,7-bis(4-hydroxy-3-methoxyphenyl)-6-heptene-3,5-dione, 1,7-bis(4-hydroxyphenyl)-1-heptene-3,5-dione, 1,7-bis(4-hydroxyphenyl)-1,4,6-heptatrien-3-one, and 1,5-bis(4-hydroxy-3-methoxyphenyl)-1,4-pentadien-3-one, 2-shogaol, 4-shogaol, 6-shogaol, 8-shogaol, 2-gingerol, 4-gingerol, 6-gingerol, 8-gingerol, ginkgolic acids, rosmanol, isorosmanol, rosmadial, carnosol, carnosic acid, epirosmanol, rosmarinic acid etc.

Compounds useful for practice of the invention include natural compounds that can be extracted or otherwise derived from Curcuma sp. as well as synthetic turmeric compounds including biologically active homologues and analogues of turmeric compounds that share anti-βA activity. Such compounds have the formula (I):

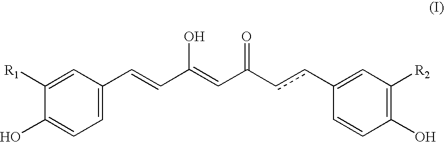

In this formula, the dotted configuration is optionally a single bond or a double bond. Generally, R₁ is selected from the group consisting of OH, OMe, OR₅₀, and X wherein R₅₀ is alkyl, alkenyl, or alkynyl, and X is F, Cl, Br, or I. Preferably, R₆ is selected from the group consisting of OH, OMe, OR₅₀ and X wherein R₅₀ is (CH₂)ₙCH₃ and n is 1-7 and X is F, Cl, Br, or I. More preferably, R₁ is selected from the group consisting of OH and OMe. Even more preferably, R₁ is OH. Even more preferably, R₁ is selected from the group consisting of OH and OMe when the dotted configuration of compound (I) is a double bond, and R₁ is selected from the group consisting of H and OH when the dotted configuration is a single bond. Generally, R₂ is selected from the group consisting of OH, OMe, OR₅₀, and X wherein R₅₀ is alkyl, alkenyl, or alkynyl, and X is F, Cl, Br, or I. Preferably, R₂ is selected from the group consisting of OH, OMe, OR₅₀ and X wherein R₅₀ is (CH₂)ₙCH₃ and n is 1-7 and X is F, Cl, Br, or I. More preferably, R₂ is selected from the group consisting of OH and OMe. Even more preferably, R₂ is OH. Even more preferably, R₂ is selected from the group consisting of OH and OMe when the dotted configuration of compound (I) is a double bond, and R₂ is H when the dotted configuration is a single bond.

Other compounds useful for practice of the invention include those of the formula (II):

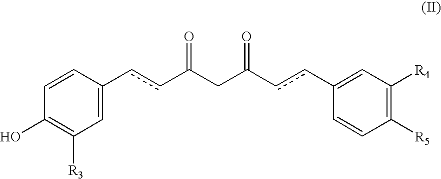

In this formula, the dotted configuration is optionally a single bond or a double bond or a triple bond. Generally, R₃ is selected from the group consisting of OH, OMe, OR₅₀, and X wherein R₅₀ is alkyl, alkenyl, or alkynyl, and X is F, Cl, Br, or I. Preferably, R₃ is selected from the group consisting of OH, OMe, OR₅₀ and X wherein R₅₀ is (CH₂)ₙCH₃ and n is 1-7 and X is F, Cl, Br, or I. More preferably, R₃ is selected from the group consisting of H, OH and OMe. Even more preferably, R₃ is OH. Even more preferably, R₃ is H. Generally; R₄ is selected from the group consisting of OH, OMe, OR₅₀, and X wherein R₅₀ is alkyl, alkenyl, or alkynyl, and X is F, Cl, Br, or I. Preferably, R₄ is selected from the group consisting of OH, OMe, OR₅₀ and X wherein R₅₀ is (CH₂)ₙCH₃ and n is 1-7 and X is F, Cl, Br, or I. More preferably, R₄ is selected from the group consisting of H, OH and OMe. Even more preferably, R₄ is H or OH. Even more preferably, R₄ is H when the first dotted configuration of compound (II) is a double bond and the second dotted configuration of compound (II) is a single bond, R₄ is H when both dotted configurations are single bonds, and R₄ is selected from the group consisting of H, OH, and OMe when both dotted configurations are double bonds. Generally, R₅ is selected from the group consisting of OH, OMe, OR₅₀, and X wherein R₅₀ is alkyl, alkenyl, or alkynyl, and X is F, Cl, Br, or I. Preferably, R₅ is selected from the group consisting of OH, OMe, OR₅₀ and X wherein R₅₀ is (CH₂)ₙCH₃ and n is 1-7 and X is F, Cl, Br, or I. More preferably, R₅ is selected from the group consisting of H, OH, and OMe. Even more preferably, R₅ is OH.

While compounds of formula (II) have been presented herein as diketones, and compounds of formula (I) have been presented as enols, those of skill in the art recognize that diketones and enols can coexist in solution as tautomers as shown below.

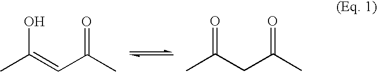

Accordingly, the invention contemplates the use and production of compounds in either tautomeric form, and as a mixture of the two forms.

A natural product compound having the following general formula was isolated from turmeric, and was found to protect cells from βA peptide-induced toxicity.

Still other turmeric-related compounds useful in practice of the invention include those of formula (III):

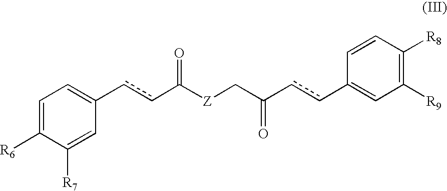

In this formula, the dotted configuration is optionally a single bond or a double bond or a triple bond. Z is a representation of isosteric variation in which Z is selected from O, S, NH, NR₆₀, where R₆₀ is alkyl, alkenyl, or alkynyl. Generally, R₆ is selected from the group consisting of OH, OMe, OR₅₀, and X wherein R₅₀ is alkyl, alkenyl, or alkynyl, and X is F, Cl, Br, or I. Preferably, R₆ is selected from the group consisting of OH, OMe, OR₅₀ and X wherein R₅₀ is (CH₂)ₙCH₃ and n is 1-7 and X is F, Cl, Br, or I. More preferably, R₆ is selected from the group consisting of OH and OMe. Even more preferably, R₆ is OH. Generally, R₇ is selected from the group consisting of OH, OMe, OR₅₀, and X wherein R₅₀ is alkyl, alkenyl, or alkynyl, and X is F, Cl, Br, or I. Preferably, R₇ is selected from the group consisting of OH, OMe, OR₅₀ and X wherein R₅₀ is (CH₂)ₙCH₃ and n is 1-7 and X is F, Cl, Br, or I. More preferably, R₇ is selected from the group consisting of H, OH and OMe. Even more preferably, R₇ is H and OH. Generally, R₈ is selected from the group consisting of OH, OMe, OR₅₀, and X wherein R₅₀ is alkyl, alkenyl, or alkynyl, and X is F, Cl, Br, or I. Preferably, R₈ is selected from the group consisting of OH, OMe, OR₅₀ and X wherein R₅₀ is (CH₂)ₙCH₃ and n is 1-7 and X is F, Cl, Br, or I. More preferably, R₈ is selected from the group consisting of H, OH, and OMe. Even more preferably, R₈ is H and OH. Generally, R₉ is selected from the group consisting of OH, OMe, OR₅₀, and X wherein R₅₀ is alkyl, alkenyl, or alkynyl, and X is F, Cl, Br, or I. Preferably, R₉ is selected from the group consisting of OH, OMe, OR₅₀ and X wherein R₅₀ is (CH₂)ₙCH₃ and n is 1-7 and X is F, Cl, Br, or I. More preferably, R₉ is selected from the group consisting of H, OH and OMe. Even more preferably, R₉ is H and OH.

The second set of compounds useful for practice of the invention include natural compounds which can be extracted on otherwise derived from Ginkgo biloba as well as synthetic Ginkgo biloba compounds including biologically active homologues and analogues of natural Ginkgo biloba compounds which share anti-βA activity. Such compounds have the formula (IV):

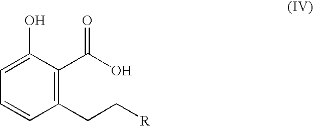

or a pharmaceutically acceptable salt or ester thereof, wherein R is selected from the group consisting of higher alkyl, higher alkenyl, and higher alkynyl.

More preferably, R is 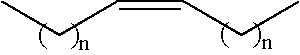
and n is 1-7. Even more preferably, R is selected from the group consisting of

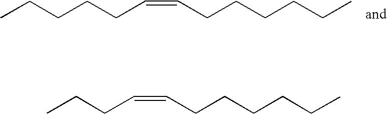

And R is also selected from the group consisting of alkyl, alkenyl, and alkynyl; for example;

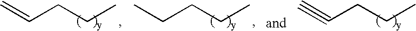

and y is 1-9, or having more than one double bond (cis or trans), or triple bond consisting of; for example;

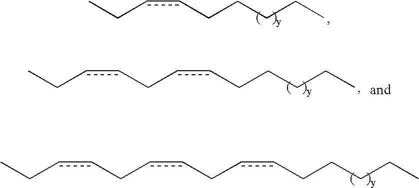

wherein the dotted line configuration is optionally a single bond (cis or trans), or a triple bond, wherein the alkyl, alkenyl, and alkynyl group is selected from ethers and/or thioethers or amines; for example; 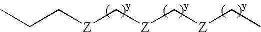
wherein z=O, S, NRₙ, where R=alkyl, alkenyl, alynyl groups; and n=1 or 2.

The third set of compounds useful for practice of the invention include natural compounds which can be extracted on otherwise derived from Zingiber sp. (ginger) as well as synthetic ginger compounds including biologically active homologues and analogues of natural ginger compounds which share anti-βA activity. Such compounds have the formula (V):

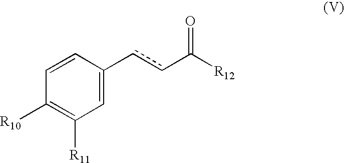

In this formula, the dotted configuration is optionally a single bond or a double bond or a triple bond. Preferably, R₁₀ is selected from the group consisting of OH, OMe, OR′, and X wherein R′ is alkyl, alkenyl, or alkynyl, and X is F, Cl, Br, or I. More preferably, R₁₀ is selected from the group consisting of OH, OMe, OR″, and X wherein R″ is (CH₂)ₙCH₃ and n is 1-7, and X is F, Cl, Br, or I. Even more preferably, R₁₀ is OH. Preferably, R₁₁ is selected from the group consisting of H, OH, OMe, and OR′ wher R′ is alkyl, alkenyl, or alkynyl. More preferably, R₁₁ is selected from the group consisting of H, OH, OMe, and OR″ wherein R″ is (CH₂)ₙCH₃ and n is 1-7. Even more preferably, R₁₁ is selected from the group consisting of H and OMe. Preferably, R₁₂ is selected from the group consisting of alkyl, alkenyl, and alkynyl. More preferably, R₁₂ is 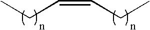
and n is 1-7. Even more preferably, R₁₂ is selected from the group consisting of 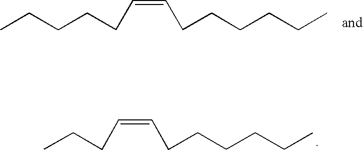
And R₁₂ is also selected from the group consisting of alkyl, alkenyl, and alkynyl; for example; 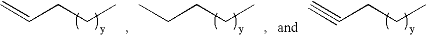
and y is 1-9, or having more than one double bond (cis or trans), or triple bond consisting of; for example; 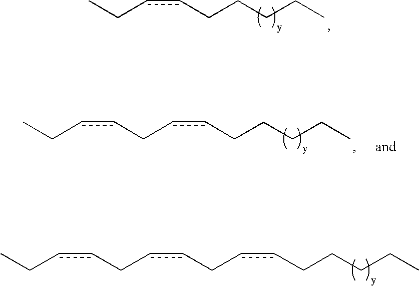
wherein the dotted line configuration is optionally a single bond (cis or trans), or a triple bond, wherein the alkyl, alkenyl, and alkynyl group is selected from ethers and/or thioethers or amines; for example; 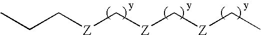
wherein z=O, S, NRₙ, where R=alkyl, alkenyl, alynyl groups; and n=1 or 2.
and compounds having a formula (VI):

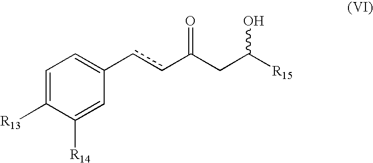

In this formula, the dotted configuration is optionally a single bond or a double bond or a triple bond. Preferably, R₁₃ is selected from the group consisting of OH, OMe, OR′, and X wherein R′ is alkyl, alkenyl, or alkynyl, and X is F, Cl, Br, or I. More preferably, R₁₃ is selected from the group consisting of OH, OMe, OR″, and X wherein R″ is (CH₂)ₙCH₃ and n is 1-7, and X is F, Cl, Br, or I. Even more preferably, R₁₃ is OH. Preferably, R₁₄ is selected from the group consisting of H, OH, OMe, and OR′ wher R′ is alkyl, alkenyl, or alkynyl. More preferably, R₁₄ is selected from the group consisting of H, OH, OMe, and OR″ wherein R″ is (CH₂)ₙCH₃ and n is 1-7. Even more preferably, R₁₄ is selected from the group consisting of H and OMe. Preferably, R₁₅ is selected from the group consisting of alkyl, alkenyl, and alkynyl. More preferably, R₁₅ is 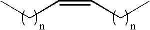
and n is 1-7. Even more preferably, R₁₅ is selected from the group consisting of 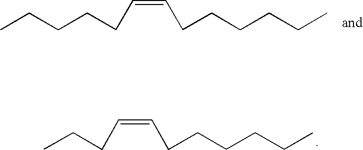
And R₁₅ is also selected from the group consisting of alkyl, alkenyl, and alkynyl; for example; 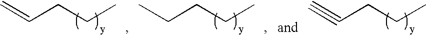
and y is 1-9, or having more than one double bond (cis or trans), or triple bond consisting of; for example; 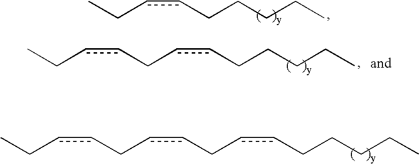
wherein the dotted line configuration is optionally a single bond (cis or trans), or a triple bond, wherein the alkyl, alkenyl, and alkynyl group is selected from ethers and/or thioethers or amines; for example; 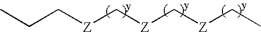
wherein z=O, S, NRₙ, where R=alkyl, alkenyl, alynyl groups; and n=1 or 2.

It is apparent from the biological results for the ginger-derived natural product compounds that the length of the side chain is important for the expression of biological activity. For example, with respect to the ginger-derived natural product compounds, compounds (11), (12), (13) and (14), the biological activity appears to improve as the compounds' side chain length increases. Thus, it is of interest to prepare analogues having different and lengthier side-chains. Preferably, shogaol compounds have side chains wherein R₁₂ has five or more carbons. More preferably, R₁₂ has nine or more carbons, and even more preferably, R₁₂ has eleven or more carbons. Furthermore, two of the synthesized shogaol analogue compounds, compounds (45) and (50), also effectively protected cells from βA peptide insult despite the fact that these compounds have different substituents than the ginger-derived natural product compounds. For example, compound (45) differs from the ginger-derived natural product compounds because it has a saturated hydrocarbon side chain, and compound (50) differs from the ginger-derived natural product compounds because it does not have a methoxy substituent. These data suggest that changing the nature of the substituents on the phenyl rings of the active compounds is of interest for the methods, pharmaceutical compositions, compounds and uses according to the invention.

As used herein, the term “alkyl” refers to a carbon chain having at least two carbons. Preferably, alkyl refers to a carbon chain having between two and twenty carbons. More preferably, alkyl refers to a carbon chain having between two and eight carbons. The term “alkenyl,” as used herein, refers to a carbon chain having at least two carbons, and at least one carbon-carbon double bond. Preferably, alkenyl refers to a carbon chain having between two and twenty carbons, and at least one carbon-carbon double bond. More preferably, the term alkenyl refers to a carbon chain having between two and eight carbons, and at least one carbon-carbon double bond. The term “alkynyl,” as used herein, refers to a carbon chain having at least two carbon atoms, and at least one carbon-carbon triple bond. Preferably, alkynyl refers to a carbon chain having between two and twenty carbon atoms, and at least one carbon-carbon triple bond. More preferably, alkynyl refers to a carbon chain having between two and eight carbon atoms, and at least one carbon-carbon triple bond.

As used herein, the term “higher alkyl” refers to a carbon chain having at least five carbon atoms. Preferably, higher alkyl refers to a carbon chain having between five and twenty carbons. More preferably, higher alkyl refers to a carbon chain having between five and twelve carbon atoms. As used herein, the term “higher alkenyl” refers to a carbon chain having at least five carbon atoms, and at least one carbon-carbon double bond. Preferably, higher alkenyl refers to a carbon chain having between five and twenty carbon atoms, and at least one carbon-carbon double bond. More preferably, higher alkenyl refers to a carbon chain having between five and twelve carbon atoms, and at least one carbon-carbon double bond. The term “higher alkynyl,” as used herein, refers to a carbon chain having at least five carbons, and at least one carbon-carbon triple bond. Preferably, higher alkynyl refers to a carbon chain having between five and twenty carbon atoms, and at least one carbon-carbon triple bond. More preferably, the term higher alkynyl refers to a carbon chain having between five and twelve carbon atoms, and at least one carbon-carbon triple bond.

The fourth set of compounds useful for practice of the invention include natural compounds which can be extracted or otherwise derived from Salvia sp. (sage) and Rosmarinus sp. (rosemary) which share anti-βA activity. Such compounds have the formula (VII):

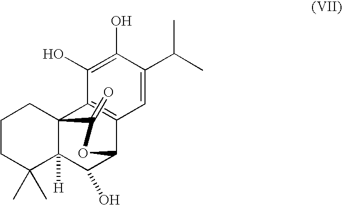

The fifth set of compounds useful for practice of the invention include natural compounds which can be extracted or otherwise derived from Salvia sp. (sage) and Rosmarinus sp. (rosemary) which share anti-βA activity. Such compounds have the formula (VIII):

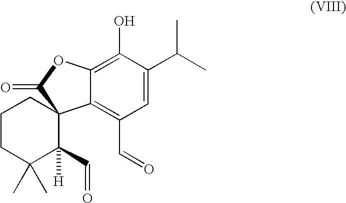

The sixth set of compounds useful for practice of the invention include natural compounds which can be extracted or otherwise derived from Salvia sp. (sage) and Rosmarinus sp. (rosemary) which share anti-βA activity. Such compounds have the formula (IX):

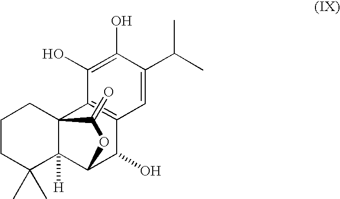

The present invention relates to the preparation and combination thereof of plant extracts that contain natural products present in Curcuma sp., Zingiber sp., Ginkgo biloba, Salvia sp., and Rosmarinus sp., which exhibit potent anti-βA activity. Specifically, the invention provides methods to prepare an extract or a combination of extracts capable of protecting neuronal cells from βA insult, and methods for treating βA-induced disease with the same.

In order to achieve the objects of the present invention, a composition is provided for treating or preventing βA-induced disease useful and suitable for the treatment or prevention of βA-induced disease, which has as major ingredients or components extracts containing natural products found in Curcuma sp., Zingiber sp., Ginkgo biloba, Salvia sp., and Rosmarinus sp. The active natural and synthetic products found in these plants are presented and discussed in preceding patent U.S. Pat. No. 6,887,898.

In one aspect, the active natural and synthetic product compounds from Curcuma sp., Zingiber sp., Ginkgo biloba, Salvia sp., and Rosmarinus sp. presented in this invention include all but are not limited to those presented and discussed in U.S. Pat. No. 6,887,898.

In a particularly preferred aspect of the present invention, the present invention provides the usage of the composition for treating or preventing βA-induced disease, in which a composition containing an extract or a combination of extracts of plants Curcuma sp., Zingiber sp., Ginkgo biloba, Salvia sp., and Rosmarinus sp.

In another aspect, the present invention provides the usage of the composition for treating or preventing βA-induced disease, in which a composition containing an extract or a combination of extracts of plants Curcuma sp., Zingiber sp., Ginkgo biloba, Salvia sp., and Rosmarinus sp., as the major constituent, in addition to members selected from brain health related therapeutic agents such as but not limited to phosphatidyl serine, docosahexaenoic acid, acetyl-L-carnitine, taurine, vitamin B12, vitamin B4, (±)-α-tocopherol, tacrine, rivastigmine, donepezil, and galantamine and the like. The compositions of the invention may also be combined with cholinesterase inhibitors used to treat Alzheimer's disease including tacrine, rivastigmine (Exelon), donepezil (Aricept), and galantamine (Reminyl) and the like.

The present invention has another object to provide a method for the preparation of the composition for treating or preventing βA-induced disease according to the present invention.

Further, the present invention has an object to provide a use of the composition according to the present invention for treating or preventing βA-induced disease.

In one aspect, the invention relates to a method for the treatment of a βA-induced disease comprising administering to a subject suffering from the βA-induced disease a therapeutically effective amount of an extract or a combination of plant extracts.

In another aspect, the invention relates to an extract or a combination of plant extracts and a pharmaceutically acceptable diluent, adjuvant, or carrier.

In another aspect, the invention relates to an extract or a combination of plant extracts, as the major constituent, in addition to a combination of brain health related therapeutic agents such as but not limited to phosphatidyl serine, docosahexaenoic acid, acetyl-L-carnitine, taurine, vitamin B12, vitamin B4 and (±)-α-tocopherol, tacrine, rivastigmine, donepezil, and galantamine and a pharmaceutically acceptable diluent, adjuvant, or carrier.

### BRIEF DESCRIPTION OF THE DRAWINGS

These and other features, aspects, and advantages of the present invention will become better understood with regard to the following description, appended claims, and accompanying drawings where:

FIG. 1 shows the structures of turmeric-derived natural product compounds that protected PC12, IMR32, and HUVEC cells from βA peptide-induced toxicity.

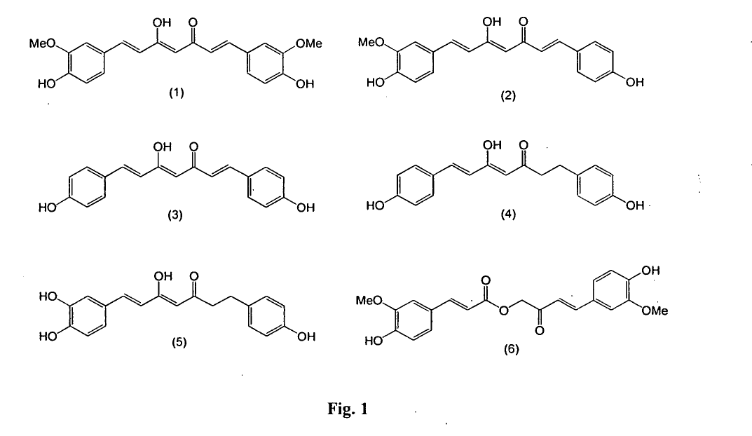

FIG. 2 shows a scheme for the synthesis of dihydro- and tetrahydro-curcuminoids.

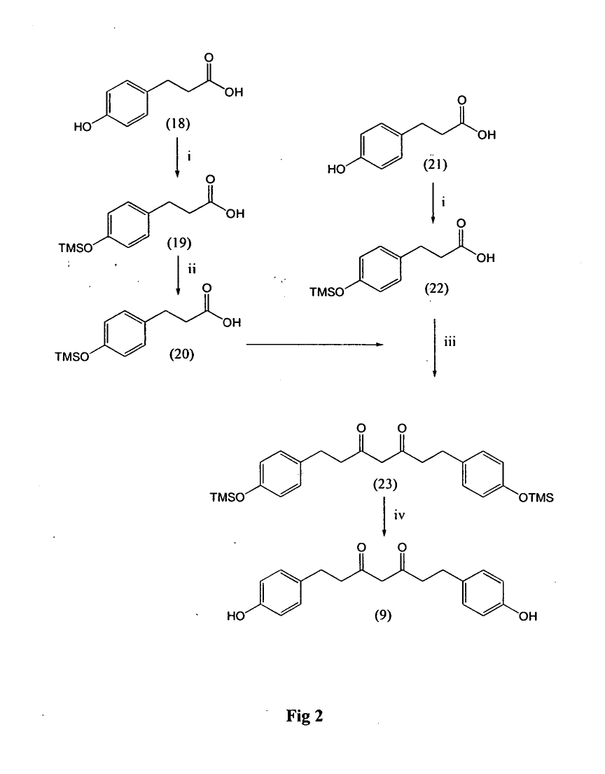

FIG. 3 shows a scheme for the synthesis of symmetric and unsymmetric curcumin analogues and related compounds.

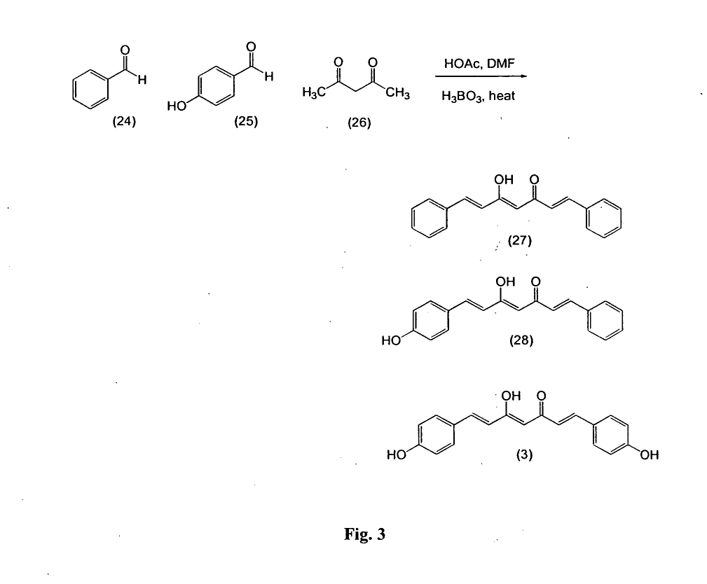

FIG. 4 shows a scheme for the synthesis of turmeric-derived natural product compound (6).

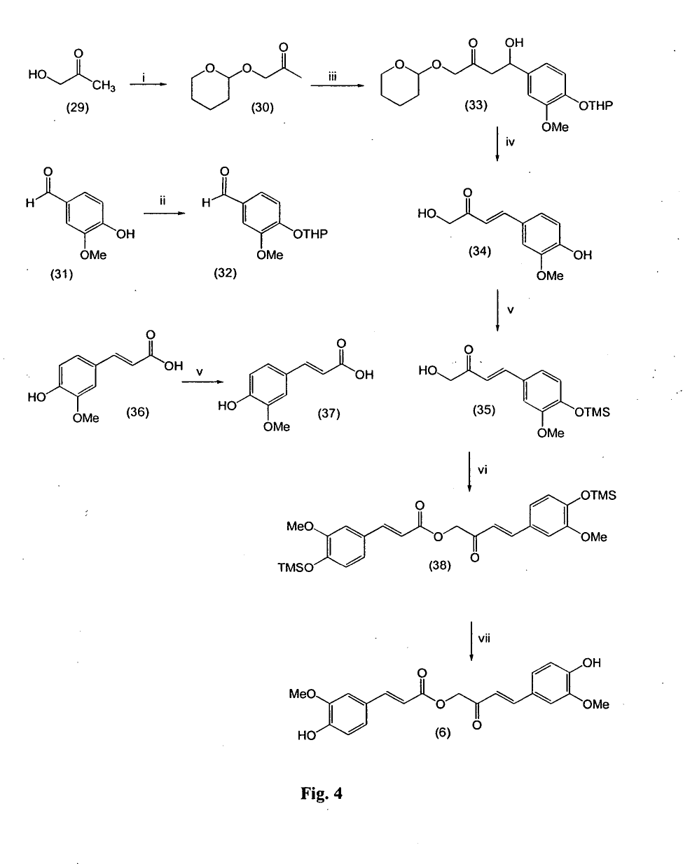

FIG. 5 shows the structures of curcuminoid compounds that have been synthetically prepared and assayed for biological activity against βA peptide-induced toxicity.

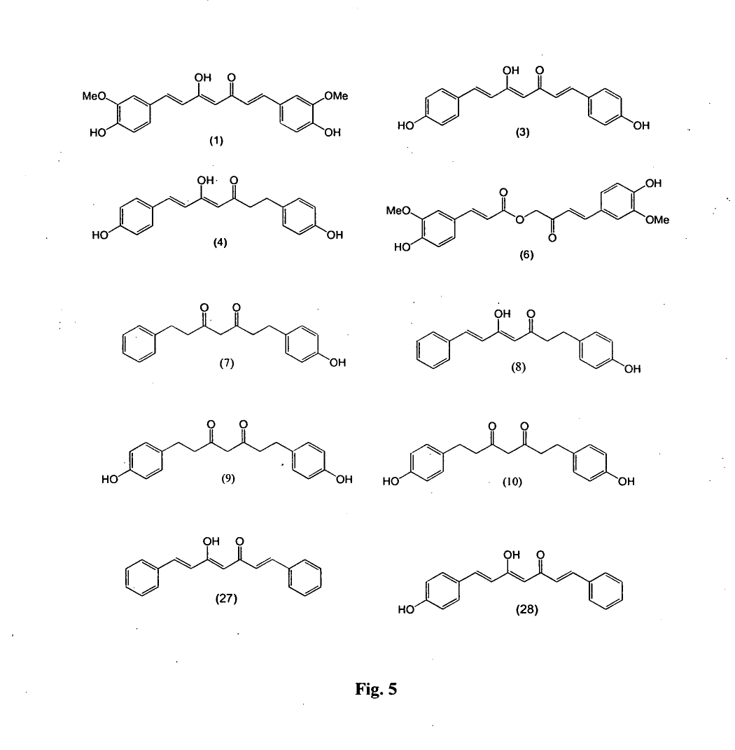

FIG. 6 shows the structures of ginger-derived natural product compounds that protected PC12, IMR32, and HUVEC cells from βA peptide-induced toxicity.

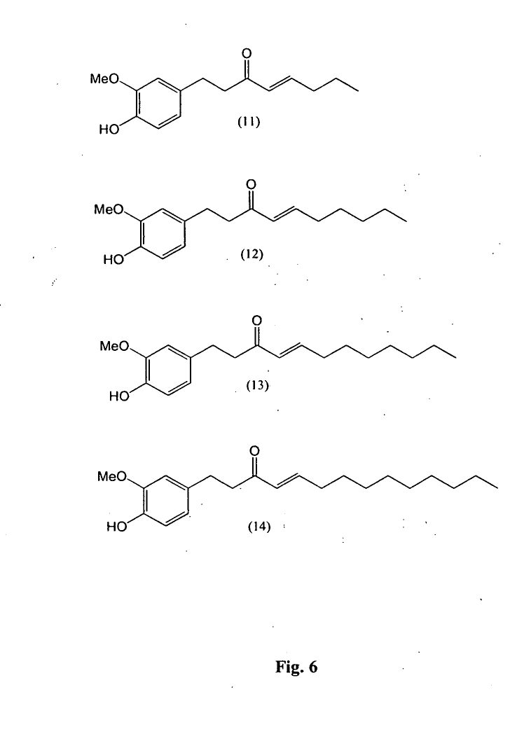

FIG. 7 shows a scheme for the synthesis of ginger-derived natural product compound (13).

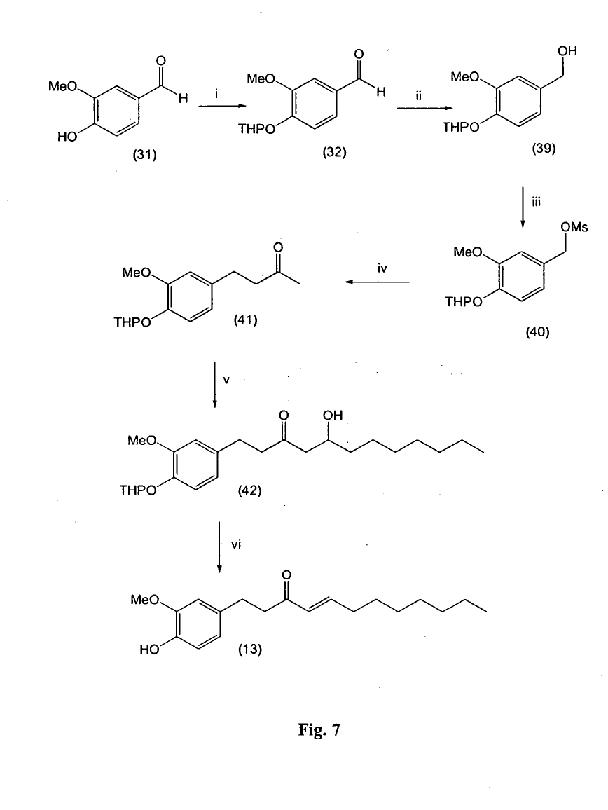

FIG. 8 shows a scheme for the synthesis of [9]-dihydroshogaol, compound (45).

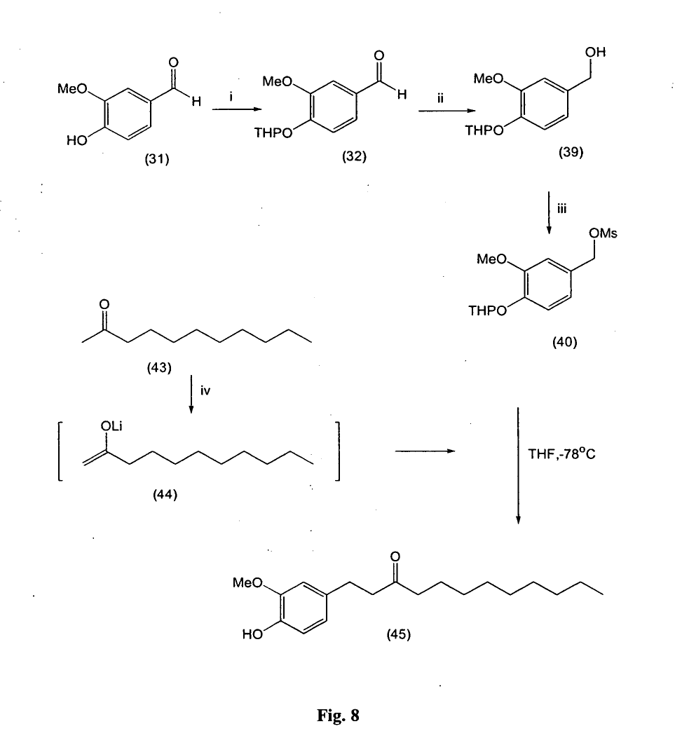

FIG. 9 shows a scheme for the synthesis of [9]-demothoxyshogaol, compound (50).

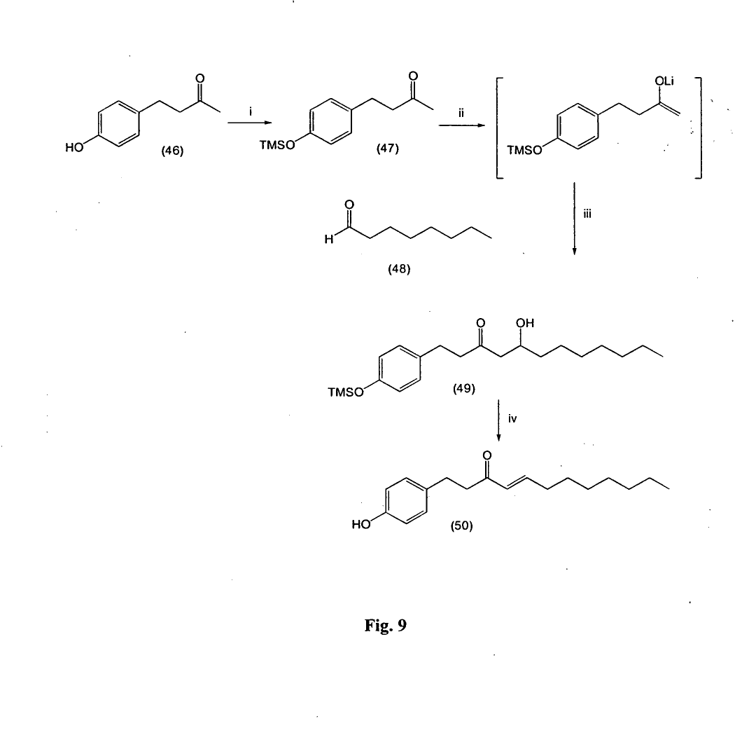

FIG. 10 shows the structures of ginkgo biloba-derived natural product compounds that protected PC12 and HUVEC cells from βA peptide-induced toxicity.

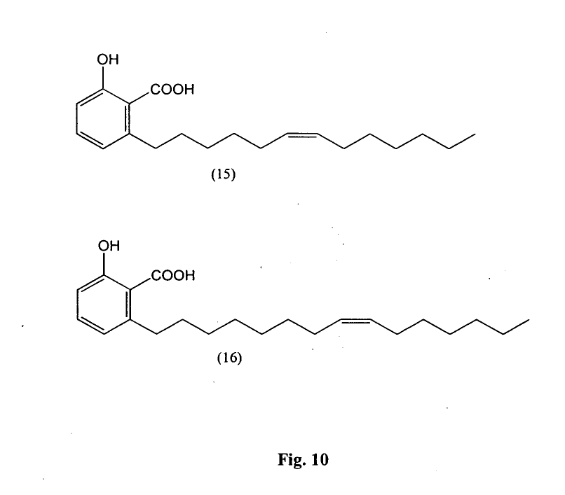

FIG. 11 shows a proposed synthesis for ginkolic acids and their analogues.

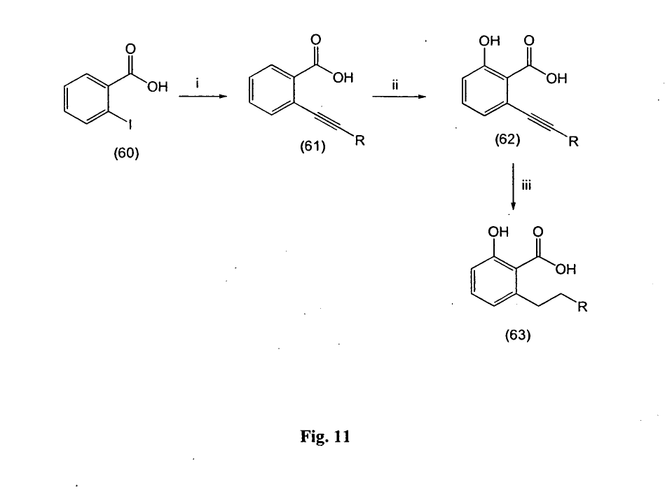

### DETAILED DESCRIPTION OF THE INVENTION

One aspect of the present invention is directed to the use of methanol and other extracts of Curcuma sp. (Zingiberaceae), Zingiber sp. (Zingiberaceae), Ginkgo biloba, Salvia sp. (Lamiaceae) and Rosmarinus sp. (Labiatae) to effectively protect cells from βA insult. The extract is obtained by pharmacologically acceptable solvent that is comprised of but not limited to methanol, ethanol, isopropyl alcohol, butanol etc. of such nature, and other nonalcoholic solvents such as dimethylsulfoxide, dimethyl formate, chloroform, dichloromethane, hexanes, petroleum ether and diethyl ether types, and in combination with water. The extracts of these plants were found to protect PC12, IMR32, and HUVEC cells from βA insult.

Methods of treating a βA-induced disease with the invention are described herein. Further, pharmaceutical compositions comprising one or more extracts of the invention and a pharmaceutically acceptable diluent, adjuvant, or carrier are provided. The use of the extracts of the invention for the manufacture of a medicament for treatment of a βA-induced disease is also disclosed herein.

The administration of the extract or combination of extracts of the invention is preferably accomplished with a pharmaceutical composition comprising a therapeutically acceptable diluent, adjuvant, or carrier. An extract or a combination of extracts according to the invention may be administered without or in conjunction with known antibiotics, surfactants, or other therapeutic agents, such as a combination of brain health related therapeutic agents such as but not limited to phosphatidyl serine, docosahexaenoic acid, acetyl-L-carnitine, taurine, vitamin B12, vitamin B4 and (±)-α-tocopherol, tacrine, rivastigmine, donepezil, and galantamine. It is contemplated that the pharmaceutical compositions of this invention can be administered to humans and other animals orally, rectally, parentally, intracisternally, intraperitoneally, topically (as by powders, ointments, or drops), bucally, intranasally, or by any other effective route of administration.

According to the methods for treatment of the present invention, βA-induced disease is treated in a subject, such as a human or lower animal, by administering to the subject a therapeutically effective amount of an extract or a combination of extracts of the invention in such amounts and for such time as is necessary to achieve the desired results. The term “βA-induced disease”, as used herein, refers to disease states that are characterized by the formulation and aggregation of βA or βA fibril deposits or plaques, such as, for example, Alzheimer's disease, and Down's syndrome.

It is contemplated that the methods for treatment in accordance with the invention encompass the treatment of subjects wherein the βA-induced disease process is ongoing but wherein the subjects do not exhibit manifest outward symptoms, and/or wherein the pathology of the disease can not be detected using presently available technologies. Furthermore, the methods for treatment of the present invention contemplate not only treating the common symptoms associated with βA-induced diseases but also treating the pathology of the disease. Thus, the methods for treatment provided herein include treating symptoms associated with βA-induced diseases, such as, for example, the memory loss and dementia associated with Alzheimer's disease, but also include preventing senile plaque formations, and/or clearing such formations. It is hypothesized that the formation of senile plaques is a regularly occurring and ongoing process in humans and other mammals. However, it is further hypothesized that the equilibrium of this process is substantially disturbed in patients affected by βA-induced diseases, resulting in the accumulation and formation of senile plaques.

As used herein, the term “therapeutically effective amount” means that amounts of an extract or a combination of extracts or a combination of extracts and other therapeutic agents of the present invention sufficient to alleviate, ameliorate, prevent, and/or clear the symptoms and/or the pathology of βA-induced disease are contemplated for administration. Accordingly, the methods for treatment of AD in accordance with the invention contemplate administration of an extract or a combination of extracts or a combination of extracts and other therapeutic agents of the invention whether βA-induced disease-like symptoms are manifested or not.

The total daily dose of an extract or a combination of extracts of this invention to be administered to a human or other mammal is preferably between 1˜200 mg/kg body weight. More preferably, the total daily dosage is between 10˜160 mg/kg body weight. Even more preferably, the total daily dosage is between 20˜100 mg/kg body weight. One skilled in the art could obtain preferred dosage ranges for the extract or combination of extracts of the invention by extrapolating from the extract or the combination of extracts' ED₅₀ values, such as, for example the ED₅₀ values presented in Table 1. It will be understood that the total daily usage of the extract or combination of extracts and composition of the present invention will be decided by the attending health professional within the scope of sound medical judgment. The specific therapeutically effective dose level for any particular patient will depend upon a variety of factors including the severity and progression of the disease, the time of administration, the route of administration, the size of the subject, the rate of excretion of the specific extract or combination of extracts employed, the duration of the treatment, the additional therapeutic agents used in combination with the specific extract or combination of extracts of the invention, and like factors well known in the medical arts.

The mechanism of action of the extract or combination of extracts or a combination of extracts and other therapeutic agents of the invention appears to involve (1) an antioxidant pathway, (2) preventing the aggregation of βA, anti-βA fibril formation, by directly binding or interacting to βA, thereby altering its structural conformation and rendering it non-toxic, (3) binding to a receptor site on the cell, thereby altering the cell function in such a way that it is protected from βA insult.

The invention can be better understood in light of the following examples which are intended as an illustration of the practice of the invention and are not meant to limit the scope of the invention in any way.

### Preparation of Extract from Curcuma longa, Zingiberaceae that Protect Cells from βA Insult

Briefly, ground turmeric was extracted with 90% methanol overnight (2×), and the solvent was removed under vacuum at 35° C. The residue was partitioned between petroleum ether/water, chloroform/water, and ethyl acetate/water, successively. As methods of extract preparation, various kinds of chromatographic techniques could be incorporated to further improve the potency of the extract. This includes gas-liquid chromatography, liquid-liquid chromatography, supercritical fluid chromatography, and column chromatography using normal phase or reverse phase stationary phase. After removing the solvent or eluent under vacuum at 35° C., the residues from each partition were screened for neuronal cell protection against βA insult using the MTT assay described below.

### Preparation of Extract from Zingiber officinale, Zingiberaceae that Protect Cells from βA Insult

Briefly, ground ginger was extracted with 90% methanol overnight (2×), and the solvent was removed under vacuum at 35° C. The residue was partitioned between petroleum ether/water, chloroform/water, and ethyl acetate/water, successively. As methods of extract preparation, various kinds of chromatographic techniques could be incorporated to further improve the potency of the extract. This includes gas-liquid chromatography, liquid-liquid chromatography, supercritical fluid chromatography, and column chromatography using normal phase or reverse phase stationary phase. After removing the solvent or eluent under vacuum at 35° C., the residues from each partition were screened for neuronal cell protection against βA insult using the MTT assay described below.

### Preparation of Extract from Ginkgo biloba that Protect Cells from βA Insult

Briefly, ground Ginkgo biloba was extracted with 90% methanol overnight (2×), and the solvent was removed under vacuum at 35° C. The residue was partitioned between petroleum ether/water, chloroform/water, and ethyl acetate/water, successively. As methods of extract preparation, various kinds of chromatographic techniques could be incorporated to further improve the potency of the extract. This includes gas-liquid chromatography, liquid-liquid chromatography, supercritical fluid chromatography, and column chromatography using normal phase or reverse phase stationary phase. After removing the solvent or eluent under vacuum at 35° C., the residues from each partition were screened for neuronal cell protection against βA insult using the MTT assay described below.

### Preparation of Extract from Salvia officinalis, Lamiaceae that Protect Cells from βA Insult

Briefly, ground sage was extracted with 90% methanol overnight (2×), and the solvent was removed under vacuum at 35° C. The residue was partitioned between petroleum ether/water, chloroform/water, and ethyl acetate/water, successively. As methods of extract preparation, various kinds of chromatographic techniques could be incorporated to further improve the potency of the extract. This includes gas-liquid chromatography, liquid-liquid chromatography, supercritical fluid chromatography, and column chromatography using normal phase or reverse phase stationary phase. After removing the solvent or eluent under vacuum at 35° C., the residues from each partition were screened for neuronal cell protection against βA insult using the MTT assay described below.

### Preparation of Extract from Rosmarinus officinalis, Labiatae that Protect Cells from βA Insult

Briefly, ground rosemary was extracted with 90% methanol overnight (2×), and the solvent was removed under vacuum at 35° C. The residue was partitioned between petroleum ether/water, chloroform/water, and ethyl acetate/water, successively. As methods of extract preparation, various kinds of chromatographic techniques could be incorporated to further improve the potency of the extract. This includes gas-liquid chromatography, liquid-liquid chromatography, supercritical fluid chromatography, and column chromatography using normal phase or reverse phase stationary phase. After removing the solvent or eluent under vacuum at 35° C., the residues from each partition were screened for neuronal cell protection against βA insult using the MTT assay described below.

### Preparation of Extracts in Different Combinations and Ratios

Briefly, the extract of each plant is mixed in certain predetermined amount (weight/weight), re-dissolved in pharmacologically acceptable solvent, and the solvent was removed under vacuum prior to bioassay.

### Screening of Cell Protection from βA Insult

The neuronal cell protection by the extract or combination of extracts was determined by observing the differences in the cell viability of βA (both 25-35 and 1-42) treated cells, βA (both 25-35 and 1-42) treated cells further including an extract or a combination of extracts according to the invention, and a DMSO control.

The degree of βA insult was measured by 3-[4,5-dimethylthiazol-2-yl]-2,5-diphenyltetrazolium bromide (MTT) reduction assay. See Kim et al., Neurosci Lett 303, 57 (2001), Park et al., J Nat Prod 65, 1227 (2002) and Kim et al., Plant Medica 68, 375 (2002). The detection of cell growth or cell death can be determined by observing the conversion of MTT to the colored product, MTT formazan, the concentration of which can be measured colorimetrically at 550 nm. See Kim et al., Neurosci Lett 303, 57 (2001).

The extract or combination of extracts' ability to protect PC12 cells from βA insult was investigated according to the published procedure. See Kim et al., Neurosci Lett 303, 57 (2001), Park et al., J Nat Prod 65, 1227 (2002) and Kim et al., Plant Medica 68, 375 (2002). For the bioassay, 90 μL of exponentially growing cells (2,000 cells per mL) were plated in 96-well tissue culture plates. Cells were incubated with βA(1-42) (2.0 μg/mL, prepared from a stock solution (1.0 mg/mL in dimethyl sulfoxide (DMSO))) and test compound at various concentrations (50, 10, 2, 0.4, and 0.08 μg/mL) for 24 hours. The final DMSO concentration was less than 1%. The extract or combination of extracts' ability to protect PC12 cells from βA(1-42) insult was determined by measuring the cell's potential to reduce MTT with respect to the treatment of cells with 1% DMSO only and the treatment of cells with 1.0 μg/mL βA(1-42) and 1% DMSO without the presence of test extract or a combination of extracts. After the incubation of cells in MTT solution (25 μL per well, 1 mg/mL stock solution) for 1 h at 37° C., 100 μL Lysing buffer (50% aqueous dimethylformamide (DMF) and 20% sodium dodecyl sulfate (SDS) at pH 4.7) was added and incubated overnight at 37° C. Optical density of the resulting solutions was colorimetrically determined at 550 nm using a microplate reader. Dose-response curves were prepared and the results were expressed as ED₅₀ values in μg/mL (Table 1). Curcumin and (±)-α-tocopherol (vitamin-E) were used as reference compounds. Bioassay using βA(25-35) was similarly performed. The extract or combination of extracts' ability to protect IMR32 and HUVEC cells from βA insult was also similarly investigated.

PC12 cells were obtained from the American Type Culture Collection (Rockville, Md.). Cells were routinely cultured on a polystyrene-coated Corning tissue culture plate (Corning, New York, N.Y.). Culture media and supplements were obtained from Life Technologies (Grand Island, N.Y.). Cells were maintained in high glucose Dulbecco's modified Eagle medium, 10% horse serum, 5% fetal calf serum, and 1% penicillin/streptomycin. See Kim et al., Neurosci Lett 303, 57 (2001), Park et al., J Nat Prod 65, 1227 (2002) and Kim et al., Plant Medica 68, 375 (2002). IMR32 human neuroblastoma cells were obtained from the American Type Culture Collection (ATCC). Normal umbilical human vein endothelial (HUVEC) cells were obtained from Clonetics (San Diego, Calif.). Cells were routinely cultured on a polystyrene-coated Corning tissue culture plate (Corning, New York, N.Y.). IMR32 cells were grown in high glucose Dulbecco's Modified Eagle Medium (DMEM), 10% horse serum, 5% fetal calf serum, and 1% penicillin/streptomycin. HUVEC cells were grown in EGM-2 Bullet Kit (Clonetics, San Diego, Calif.). For the bioassay using βA(25-35), 100 μl of exponentially growing IMR32 and HUVEC cells (2,000 and 500 cells per ml, respectively) were plated in 96-well tissue culture plates. A different number of cells per ml was used for the experiment because of the cell size difference of HUVEC cells with respect to IMR32 cells and PC12 cells. Both βA(25-35) and βA(1-42) were purchased from Bachem California (Torrance, Calif.). MTT and other chemicals were purchased from Sigma/Aldrich (St. Louis, Mo.).

ED₅₀ values reflect the results from the MTT reduction assay, and represent the sample concentration that is required to achieve 50% cell viability, a mid-point between the values obtained from 1% DMSO only treatment and βA(25-35) (1.0 μg/ml) and 1% DMSO treatment. The samples that gave values as determined by the MTT reduction assay less than or equal to that of βA only treated wells were considered cytotoxic or without desired activity, and are labeled “toxic”.

### The Merit of Combination of Extracts over Single Extract

The results show that combination of extracts wt/wt showed remarkable synergistic effect in protecting cells from βA insult with respect to that of single extract (see table 1).

TABLE 1
Cell protection against βA insult by an extract or a combination of extracts.

| | | | IMR32 | | | |
|---|---|---|---|---|---|---|
| | PC12 | | ED₅₀(μg/ml) | | HUVEC | |
| extract (ratio) | βA(25-35) | βA(1-42) | βA(25-35) | βA(1-42) | βA(25-35) | βA(1-42) |
| T | 13.7 | 14.3 | 12.4 | 15.2 | 17.7 | 16.2 |
| G | 13.9 | 12.2 | 14.5 | 15.7 | 14.2 | 12.8 |
| Gk | 14.6 | 13.3 | 15.2 | 14.6 | 15.5 | 16.7 |
| S | 15.8 | 17.2 | 13.3 | 14.7 | 17.8 | 16.6 |
| R | 14.8 | 15.5 | 18.2 | 17.3 | 18.6 | 16.4 |
| T/G (50/50) | 11.4 | 9.8 | 10.6 | 9.5 | 11.1 | 11.7 |
| T/Gk (50/50) | 10.2 | 11.1 | 9.8 | 12.9 | 10.4 | 9.7 |
| G/Gk (50/50) | 9.5 | 9.4 | 10.6 | 11.1 | 9.9 | 10.2 |
| T/G/Gk (33/33/33) | 4.4 | 5.2 | 5.1 | 4.9 | 5.2 | 4.8 |
| T/G/S (33/33/33) | 5.6 | 5.9 | 6.4 | 6.5 | 5.8 | 6.9 |
| T/G/S/R (33/32/16/16) | 4.8 | 5.7 | 5.5 | 5.3 | 4.6 | 5.3 |
| curcumin | 6.7 | 7.1 | 7.4 | 6.3 | 6.2 | 6.8 |
| α-tocopherol | >50 | >50 | >50 | >50 | >50 | >50 |

T represents turmeric extract;
G represents ginger extract;
Gk represents ginkgo extract;
S represents sage extract;
R represents rosemary extract;
ED₅₀ represent the sample concentration that is required to achieve 50% cell viability, a mid-point between the values obtained from 1% DMSO only treatment and βA and 1% DMSO treatment.
Note:
The tests were performed in triplets on three different dates. Data are mean ± SEM from nine determinations. P < 0.05 (student's t-test).

Disease (AD), and Down's syndrome. The invention also provides methods of treating beta-Amyloid induced ocular disease including, in particular, glaucoma and age-related macular degeneration (AMD) according to the methods described in co-owned and copending U.S. patent application Ser. No. 11/287,080 filed Nov. 23, 2005 [Attorney Docket No. 30443/41270] entitled “Methods for treatment of Beta-Amyloid Protein-Induced Ocular Disease” the disclosure of which is hereby incorporated by reference.

According to one aspect of the invention an extract or a combination of extracts containing natural compounds found in particular plants (as well as synthetic analogues and homologues thereof) as the major ingredients or components may be administered to protect cells from βA insult. Natural compounds that are suitable for use with the invention include, but are not limited to 4″-(3″′-methoxy-4″′-hydroxyphenyl)-2″-oxo-3″-enebutanyl 3-(3′-methoxy-4′hydroxyphenyl)propenoate (calebin-A) and 1,7-bis(4-hydroxy-3-methoxyphenyl)-1,4,6-heptatrien-3-one, and seven known compounds, 1,7-bis(4-hydroxy-3-methoxyphenyl)-1,6-heptadiene-3,5-dione (curcumin), 1-(4-hydroxy-3-methoxyphenyl)-7-(4-hydroxyphenyl)-1,6-heptadiene-3,5-dione (demethoxycurcumin), 1,7-bis(4-hydroxyphenyl)-1,6-heptadiene-3,5-dione (bisdemethoxycurcumin), 1-hydroxy-1,7-bis(4-hydroxy-3-methoxyphenyl)-6-heptene-3,5-dione, 1,7-bis(4-hydroxyphenyl)-1-heptene-3,5-dione, 1,7-bis(4-hydroxyphenyl)-1,4,6-heptatrien-3-one, and 1,5-bis(4-hydroxy-3-methoxyphenyl)-1,4-pentadien-3-one, 2-shogaol, 4-shogaol, 6-shogaol, 8-shogaol, 2-gingerol, 4-gingerol, 6-gingerol, 8-gingerol, ginkgolic acids, rosmanol, isorosmanol, rosmadial, carnosol, carnosic acid, epirosmanol, rosmarinic acid etc.

Compounds useful for practice of the invention include natural compounds that can be extracted or otherwise derived from Curcuma sp. as well as synthetic turmeric compounds including biologically active homologues and analogues of turmeric compounds that share anti-βA activity. Such compounds have the formula (I):

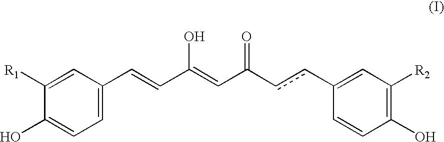

TABLE 3
Cell protection against βA insult by representative compounds
from plants and combination of compounds thereof.

| | | | IMR32 | | | |
|---|---|---|---|---|---|---|
| | PC12 | | ED₅₀(μg/ml) | | HUVEC | |
| Compound (ratio) | βA(25-35) | βA(1-42) | βA(25-35) | βA(1-42) | βA(25-35) | βA(1-42) |
| RS-7 | 2.3 | 3.1 | 2.9 | 3.4 | 3.2 | 3.5 |
| RS-8 | 11.6 | 13.3 | 15.2 | 14.6 | 15.5 | 16.7 |
| RS-9 | 33.2 | 35.4 | 38.3 | 35.5 | 31.0 | 34.2 |
| 6-shogaol [6S] | 5.4 | 5.7 | 6.3 | 6.1 | 5.9 | 5.5 |
| Curcumin [Cur] | 6.7 | 7.1 | 7.4 | 6.3 | 6.2 | 6.8 |
| 6S/Cur/RS-7 (33 [symbol] | 2.8 | 1.9 | 2.4 | 2.7 | 2.9 | 2.6 |
| 6S/Cur/RS-8 (33 [symbol] | 5.6 | 4.3 | 4.7 | 5.1 | 4.7 | 3.8 |
| 6S/Cur/R(33/33/33) | 2.6 | 2.8 | 3.2 | 3.4 | 3.1 | 3.9 |
| α-tocopherol | >50 | >50 | >50 | >50 | >50 | >50 |

6S represents 6-shogaol
Cur represents curcumin
RS-7 represents compound (VII) from sage and rosemary
RS-8 represents compound (VIII) from sage and rosemary
RS-9 represents compound (IX) from sage and rosemary
S represents sage extract.
R represents rosemary extract.
ED₅₀ represents the sample concentration that is required to achieve 50% cell viability, a mid-point between the values obtained from 1% DMSO only treatment and βA and 1% DMSO treatment.
Note:
The tests were performed in triplets on three different dates.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. A method for the treatment of a beta-Amyloid protein-induced disease comprising administering to a subject suffering from a beta-Amyloid protein induced disease a therapeutically effective amount of a composition comprising a different member selected from each at least two of a) a natural or synthetic turmeric compound having anti-βA peptide activity; b) a natural or synthetic ginkgo biloba compound having anti-βA peptide activity; and c) a natural or synthetic ginger compound having anti-βA peptide activity; d) a natural or synthetic sage compound having anti-βA peptide activity; and e) a natural or synthetic rosemary compound having anti-βA peptide activity.

2. The method of claim 1 comprising administering to a subject suffering from the beta-Amyloid protein-induced disease a therapeutically effective amount of a composition comprising a different member selected from at least two of:
a) a compound having the formula (I): 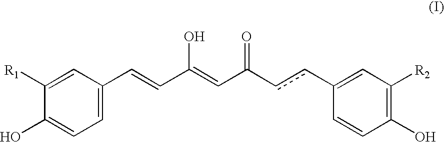
or a compound having the formula (II): 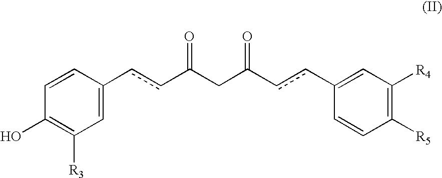
or a compound having the formula (III): 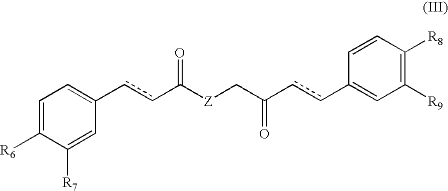
or pharmaceutically acceptable salts or esters thereof, wherein: the dotted configuration [symbol] is optionally a single bond or a double bond or a triple bond;
Z is a representation of isosteric variation in which Z is selected from O, S, NH, NR₆₀, where R₆₀ is alkyl, alkenyl, or alkynyl;
R₁ is selected from the group consisting of H, OH, OMe, and OR₅₀ wherein R₅₀ is alkyl, alkenyl, or alkynyl;
R₂ is selected from the group consisting of H, OMe, and OR₅₀ wherein R₅₀ is alkyl, alkenyl, or alkynyl;
R₃ is selected from the group consisting of H, OMe, and OR₅₀ wherein R₅₀ is alkyl, alkenyl, or alkynyl;
R₄ is selected from the group consisting of H, OH, OMe, and OR₅₀ wherein R₅₀ is alkyl, alkenyl, or alkynyl;
R₅ is selected from the group consisting of H, OH, OMe, OR₅₀, and X wherein R₅₀ is alkyl, alkenyl, or alkynyl, and X is F, Cl, Br, or I;
R₆ is selected from the group consisting of OH, OMe, OR₅₀, and X wherein R₅₀ is alkyl, alkenyl, or alkynyl, and X is F, Cl, Br, or I;
R₇is selected from the group consisting of H, OMe, and OR₅₀ wherein R₅₀ is alkyl, alkenyl, or alkynyl;
R₈ is selected from the group consisting of OH, OMe, OR₅₀ and X wherein R₅₀ is alkyl, alkenyl, or alkynyl, and X is F, Cl, Br, or I; and
R₉ is selected from the group consisting of H, OMe and OR₅₀ wherein R₅₀ is alkyl, alkenyl, or alkynyl;
b) a compound having the formula (IV): 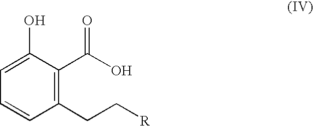
or a pharmaceutically acceptable salt or ester thereof, wherein:
R is selected from the group consisting of higher alkyl, higher alkenyl, and higher alkynyl
c) a compound having the formula (V): 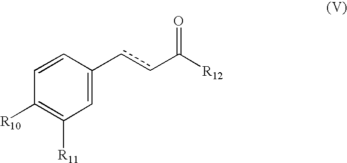
or a pharmaceutically acceptable salt or ester thereof, wherein:
the dotted configuration [symbol] is optionally a single bond or a double bond;
R₁₀ is selected from the group consisting of OH, OMe, OR′, and X wherein R′ is alkyl, alkenyl, or alkynyl, and X is F, Cl, Br, or I;
R₁₁ is selected from the group consisting of H, OH, OMe, and OR′ wherein R′ is alkyl, alkenyl, or alkynyl; and
R₁₂ is selected from the group consisting of alkyl, alkenyl, and alkynyl;
d) a compound having a formula (VI): 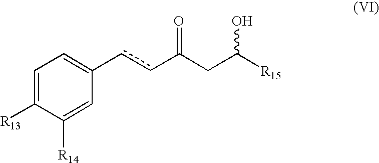
or a pharmaceutically acceptable salt or ester thereof, wherein:
the dotted configuration [symbol] is optionally a single bond or a double bond or a triple bond;
R₁₃ is selected from the group consisting of OH, OMe, OR′, and X wherein R′ is alkyl, alkenyl, or alkynyl, and X is F, Cl, Br, or I;
R₁₄ is selected from the group consisting of H, OH, OMe, and OR′ wherein R′ is alkyl, alkenyl, or alkynyl; and
R₁₅ is selected from the group consisting of alkyl, alkenyl, and alkynyl;
e) a compound having the formula (VII): 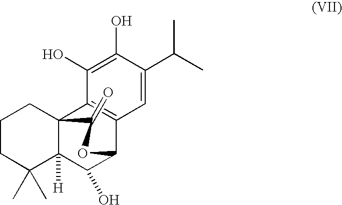
f) a compound having the formula (VIII): 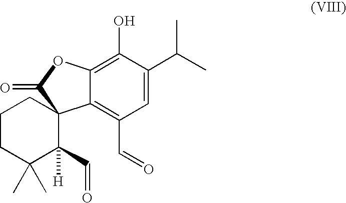
g) a compound having the formula (IX):

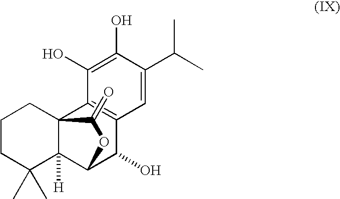

3. The method of claim 1 wherein a) is an extract from Curcuma sp. Zingiberaceae.

4. The method of claim 1 wherein b) is an extract from Ginkgo biloba Ginkgoaceae.

5. The method of claim 1 wherein c) is an extract from Zingiber sp. Zingiberaceae.

6. The method of claim 1 wherein d) is an extract from Salvia sp. Lamiaceae.

7. The method of claim 1 wherein e) is an extract from Rosmarinus sp. Labiatae.

8. The method of claim 2 wherein R is 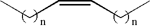
and n is 1-7.

9. The method of claim 8 wherein R is selected from the group consisting of

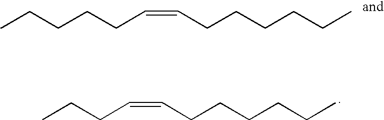

10. The method of claim 2 wherein R₁₂ is 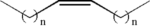
and n is 1-7.

11. The method of claim 10 wherein R₁₂ is selected from the group consisting of

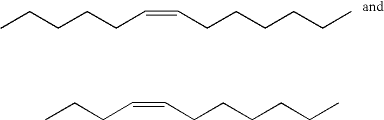

12. The method of claim 2 wherein R₁₅ is 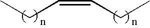
and n is 1-7.

13. The method of claim 12 wherein R₁₅ is selected from the group consisting of

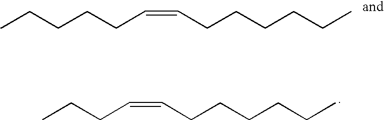

14. The method of claim 1 wherein the beta-Amyloid induced disease induces cytotoxicity.

15. The method of claim 1 wherein the subject is suffering from Alzheimer's disease.

16. The method of claim 1 in which the beta-Amyloid protein-induced cytotoxicity is neurotoxicity.

17. The method according to claim 2 wherein a purified and isolated compound selected from the group consisting of formula (I), (II), (III), (IV), (V), (VI), (VII), (VIII), and (IX) is administered.

18. The method of claim 1 wherein the composition further comprises one or more ingredients selected from the group consisting of phosphatidyl serine, docosahexaenoic acid, acetyl-L-carnitine, taurine, vitamin B12, vitamin B4, (±)-α-tocopherol, tacrine, rivastigmine, donepezil, and galantamine.

19. A pharmaceutical composition comprising at least one different member from at least two of a) a natural or synthetic turmeric compound having anti-βA peptide activity; b) a natural or synthetic ginkgo biloba compound having anti-βA peptide activity; c) a natural or synthetic ginger compound having anti-βA peptide activity; d) a natural or synthetic sage compound having anti-βA peptide activity; e) a natural or synthetic rosemary compound having anti-βA peptide activity.

20. The composition of claim 19 comprising at least one different member selected from at least two of:
a) a compound having the formula (I): 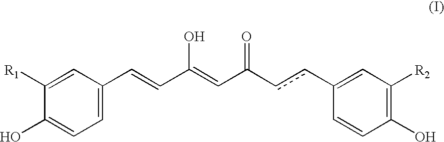
or a compound having the formula (II): 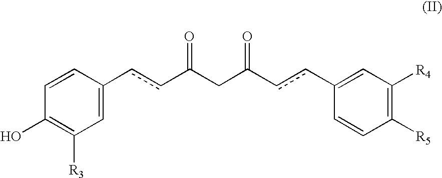
or a compound having the formula (III): 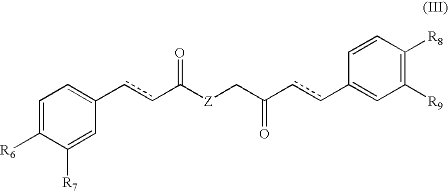
or pharmaceutically acceptable salts or esters thereof, wherein:
the dotted configuration [symbol] is optionally a single bond or a double bond or a triple bond;
Z is a representation of isosteric variation in which Z is selected from O, S, NH, NR₆₀, where R₆₀ is alkyl, alkenyl, or alkynyl;
R₁ is selected from the group consisting of H, OH, OMe, and OR₅₀ wherein R₅₀ is alkyl, alkenyl, or alkynyl;
R₂ is selected from the group consisting of H, OMe, and OR₅₀ wherein R₅₀ is alkyl, alkenyl, or alkynyl;
R₃ is selected from the group consisting of H, OMe, and OR₅₀ wherein R₅₀ is alkyl, alkenyl, or alkynyl;
R₄ is selected from the group consisting of H, OH, OMe, and OR₅₀ wherein R₅₀ is alkyl, alkenyl, or alkynyl;
R₅ is selected from the group consisting of H, OH, OMe, OR₅₀, and X wherein R₅₀ is alkyl, alkenyl, or alkynyl, and X is F, Cl, Br, or I;
R₆ is selected from the group consisting of OH, OMe, OR₅₀, and X wherein R₅₀ is alkyl, alkenyl, or alkynyl, and X is F, Cl, Br, or I;
R₇ is selected from the group consisting of H, OMe, and OR₅₀ wherein R₅₀ is alkyl, alkenyl, or alkynyl;
R₈ is selected from the group consisting of OH, OMe, OR₅₀ and X wherein R₅₀ is alkyl, alkenyl, or alkynyl, and X is F, Cl, Br, or I; and
R₉ is selected from the group consisting of H, OMe and OR₅₀ wherein R₅₀ is alkyl, alkenyl, or alkynyl;
b) a compound having the formula (IV): 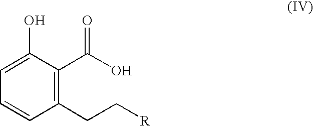
or a pharmaceutically acceptable salt or ester thereof, wherein:
R is selected from the group consisting of higher alkyl, higher alkenyl, and higher alkynyl
c) a compound having the formula (V): 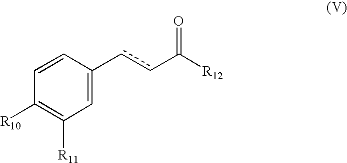
or a pharmaceutically acceptable salt or ester thereof, wherein:
the dotted configuration [symbol] is optionally a single bond or a double bond;
R₁₀ is selected from the group consisting of OH, OMe, OR′, and X wherein R′ is alkyl, alkenyl, or alkynyl, and X is F, Cl, Br, or I;
R₁₁ is selected from the group consisting of H, OH, OMe, and OR′ wherein R′ is alkyl, alkenyl, or alkynyl; and
R₁₂ is selected from the group consisting of alkyl, alkenyl, and alkynyl;
d) a compound having a formula (VI): 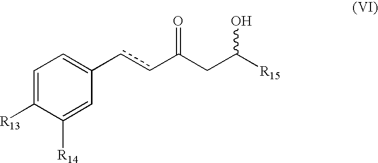
or a pharmaceutically acceptable salt or ester thereof, wherein:
the dotted configuration [symbol] is optionally a single bond or a double bond or a triple bond;
R₁₃ is selected from the group consisting of OH, OMe, OR′, and X wherein R′ is alkyl, alkenyl, or alkynyl, and X is F, Cl, Br, or I;
R₁₄ is selected from the group consisting of H, OH, OMe, and OR′ wherein R′ is alkyl, alkenyl, or alkynyl; and
R₁₅ is selected from the group consisting of alkyl, alkenyl, and alkynyl, and
e) a compound having the formula (VII), (VIII) or (IX):

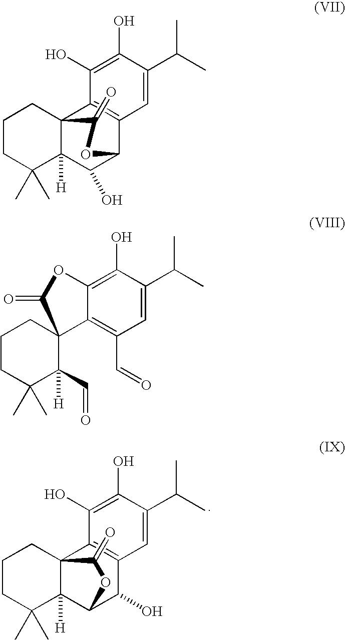

21. The composition of claim 20 wherein a) is an extract from Curcuma sp. (Zingiberaceae).

22. The composition of claim 20 wherein b) is an extract from Ginkgo biloba (Ginkgoaceae).

23. The composition of claim 20 wherein c) is an extract from Zingiber sp. (Zingiberaceae).

24. The composition of claim 20 wherein d) is an extract from Salvia sp. (Lamiaceae).

25. The composition of claim 20 wherein e) is an extract from Rosmarinus sp. (Labiatae).

26. The composition of claim 20 wherein a purified and isolated compound selected from the group consisting of formula (I), (II), (III), (IV), (V), (VI), (VII), (VIII) and (IX) is administered.

27. The composition of claim 20, which comprises an extract from one or more of Curcuma sp. (Zingiberaceae), from Zingiber sp. (Zingiberaceae), Ginkgo biloba (Ginkgoaceae), Salvia sp. (Lamiaceae), or Rosmarinus sp. (Labiatae) wherein said extract is prepared by immersing said plant in said solvent at a ratio from about 1% to about 100% plant weight to solvent volume.

28. The composition of claim 20, wherein said extract is further concentrated.

29. The composition of claim 20 wherein at least one extract is prepared by the steps comprising:
a) obtaining an extract by immersing a plant Curcuma sp. or Zingiber sp. or Ginkgo biloba, or Salvia sp., or Rosmarinus sp. in a pharmacologically acceptable solvent; and
b) concentrating said extract; and
c) partitioning of said plant: extract using a combination of a pharmacologically acceptable solvent and water; and
d) concentrating said partitioned extract.

30. The composition of claim 20 further comprising one or more ingredients selected from the group consisting of phosphatidyl serine, docosahexaenoic acid, acetyl-L-carnitine, taurine, vitamin B12, vitamin B4, (±)-α-tocopherol, tacrine, rivastigmine, donepezil, and galantamine.

31. A method for the preparation of a composition for the treatment of a beta-Amyloid protein-induced disease comprising the steps of obtaining extracts from each of Curcuma sp. (Zingiberaceae), Zingiber sp. (Zingiberaceae), Ginkgo biloba (Ginkgoaceae), Salvia sp. (Lamiaceae), and Rosmarinus sp. (Labiatae) which extracts have activity neutralizing beta-Amyloid cytotoxicity and combining each of said extracts to form a pharmaceutical composition.

32. A method for the treatment of beta-Amyloid protein-induced disease comprising administering to a subject suffering from a beta-Amyloid protein induced disease a therapeutically effective amount of a composition comprising a member selected from:
a compound having the formula (VII): 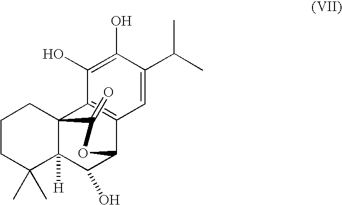
a compound having the formula (VIII): 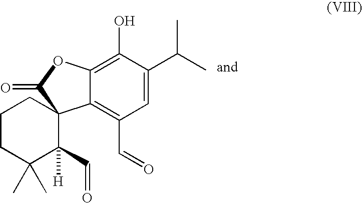
a compound having the formula (IX):

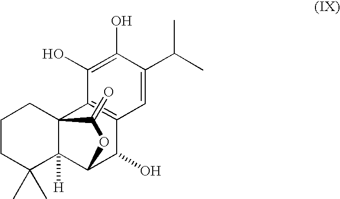

33. The method of claim 32 wherein the beta-Amyloid induced disease induces cytotoxicity.

34. The method of claim 32 wherein the subject is suffering from Alzheimer's disease.

35. The method of claim 32 in which the beta-Amyloid protein-induced cytotoxicity is neurotoxicity.
